(19) United States
(12) Patent Application Publication
(10) Pub. No.: US 2016/0374638 A1
(43) Pub. Date: Dec. 29, 2016

(54) RADIOLOGICAL IMAGING APPARATUS AND RADIOLOGICAL IMAGING SYSTEM

(71) Applicant: Konica Minolta, Inc., Tokyo (JP)

(72) Inventor: TETSUO KUBOTA, Tokyo (JP)

(21) Appl. No.: 15/192,269

(22) Filed: Jun. 24, 2016

(30) Foreign Application Priority Data
Jun. 24, 2015 (JP) 2015-126102

(51) Int. Cl. A61B 6/00

(52) U.S. Cl. CPC A61B 6/545; A61B 6/5294; A61B 6/4266

(57) ABSTRACT

A radiological imaging apparatus includes: a plurality of radiation detecting elements configured to generate charge in accordance with a dose of radiation emitted; a control unit configured to conduct control to perform an image data generation process to generate image data by reading the charge; and a storage unit configured to store the image data, wherein the control unit is capable of switching an imaging mode between an imaging enabled mode and an imaging disabled mode, when at least one kind of information of patient information and imaged site information is input, the control unit switches the imaging mode from the imaging disabled mode to the imaging enabled mode, and after attaching the information to the image data and storing the image data into the storage unit, the control unit switches the imaging mode from the imaging enabled mode to the imaging disabled mode.

The entire disclosure of Japanese Patent Application No. 2015-126102 filed on Jun. 24, 2015 including description, claims, drawings, and abstract are incorporated herein by reference in its entirety.

### BACKGROUND OF THE INVENTION

Field of the Invention

The present invention relates to a radiological imaging apparatus and a radiological imaging system.

Description of the Related Art

Various kinds of radiological imaging apparatuses (or flat panel detectors) have been developed recently, and such a radiological imaging apparatus has radiation detecting elements two-dimensionally arranged therein, and reads image data formed with the charge generated in the radiation detecting elements in accordance with radiation that is emitted from an irradiation device and penetrates through an object. These radiological imaging apparatuses are being used for imaging at facilities such as hospitals. Also, portable radiological imaging apparatuses that can be carried around have been developed recently, and been put into practical use. Such a portable radiological imaging apparatus has a sensor panel or the like provided in a housing, and radiation detecting elements and the like are formed on the sensor panel.

At present, such a portable radiological imaging apparatus is normally designed to perform an imaging operation under the control of a console that is formed with a computer or the like. Specifically, a console is designed to activate a radiological imaging apparatus, switch the radiological imaging apparatus between a power consuming state (or a power saving state (also called a sleep state or the like) and an imaging enabled state (also called a wake-up state or the like), cause the radiological imaging apparatus to transmit image data or the like every time imaging is performed, and associate a generated radiological image with (or link a generated radiological image to) the corresponding imaging order information containing an instruction related to the radiological imaging (see WO 2011/142157 A, for example).

In a case where a console controls operation of a radiological imaging apparatus, the console normally transmits information such as imaging order information related to the imaging to be performed, to the radiological imaging apparatus. The radiological imaging apparatus is controlled to perform the imaging in the sequence according to the information. Image data is then transmitted from the radiological imaging apparatus in the same sequence as the sequence of imaging, to the console.

When radiological images are associated with the imaging order information as described above, the console sequentially associates the imaging order information with the radiological images that have been generated in accordance with the image data or the like and been transmitted from the radiological imaging apparatus. With this structure, any radiological image is not associated with wrong imaging order information, and radiological images can be accurately associated with the corresponding imaging order information.

Meanwhile, as disclosed in JP 2010-137059 A, radiological imaging apparatuses of a different type have been known. A radiological imaging apparatus of this type performs imaging free of the control of any console, and stores obtained image data in a storage unit provided in the radiological imaging apparatus. A cable is connected to a radiological imaging apparatus having such a structure, or the storage unit is removed from the radiological imaging apparatus and is connected to a console, so that obtained image data can be transmitted from the radiological imaging apparatus to the console.

In a case where such a radiological imaging apparatus performs imaging free of the control of a console, an operator who is a radiological technologist or the like can conduct imaging freely, without the imaging order information or the like imposing any restrictions on the imaging operation. However, the console needs to associate the imaging order information with the radiological images generated in accordance with the obtained image data or the like.

In a case where an operator who is a imaging order information or the like associates radiological images with imaging order information, problems might occur. For example, a radiological image might be associated with wrong imaging order information, or it might be unclear which imaging order information corresponds to which radiological image.

In view of this, there are cases where a radiological imaging apparatus is designed to display the number (indicating what number imaging) assigned to image data stored in an internal storage unit (the number is written as accompanying information in the header or the like of the image data). An operator who is a radiological technologist or the like takes a note by looking at the displayed number and writing down the number on the irradiation record corresponding to the current imaging, or takes a note by writing down the number and associating the number with the patient or the imaged site (in a case where no irradiation record is available). In this manner, a radiological image is associated with the imaging order information created before or after the imaging, in accordance with the number and the note attached to the radiological image generated in accordance with the image data (with the number written in the header or the like).

However, with such a structure, an operator who is a radiological technologist or the like needs to look at the number displayed on the radiological imaging apparatus, write down the number, and associate the patient or the imaged site with the number. Since a person (an operator) needs to carry out many procedures, the possibility of a human error is not effectively reduced.

Associating a radiological image with wrong imaging order information might lead to misdiagnosis. For example, a patient might be misdiagnosed with an injury or a disease the patient is not inflicted with, or a patient might be misdiagnosed as having recovered from an injury or a disease the patient is still suffering from.

### SUMMARY OF THE INVENTION

The present invention has been made in view of the above problems, and an object of the invention is to provide a radiological imaging apparatus and a radiological imaging system that are capable of substantially reducing the possibility of a human error even in a case where imaging is performed in the radiological imaging apparatus free of the control of a console.

To achieve the abovementioned object, according to an aspect, a radiological imaging apparatus reflecting one aspect of the present invention comprises: a plurality of radiation detecting elements two-dimensionally arranged and configured to generate charge in accordance with a dose of radiation emitted; a control unit configured to conduct control to perform an image data generation process to generate image data by reading the charge from each of the radiation detecting elements; and a storage unit configured to store the image data, wherein the control unit is capable of switching an imaging mode between an imaging enabled mode in which the image data generation process is enabled and an imaging disabled mode in which the image data generation process is disabled, when at least one kind of information of patient information and imaged site information is input, the control unit switches the imaging mode from the imaging disabled mode to the imaging enabled mode, and after attaching the information to the image data generated by imaging performed in the imaging enabled mode and storing the image data into the storage unit, the control unit switches the imaging mode from the imaging enabled mode to the imaging disabled mode.

To achieve the abovementioned object, according to an aspect, a radiological imaging system reflecting one aspect of the present invention comprises: the abovementioned radiological imaging apparatus; an input unit configured to input the at least one kind of information of the patient information and the imaged site information to the radiological imaging apparatus; and an irradiation device configured to emit radiation to the radiological imaging apparatus.

### BRIEF DESCRIPTION OF THE DRAWINGS

The above and other objects, advantages and features of the present invention will become more fully understood from the detailed description given hereinbelow and the appended drawings which are given by way of illustration only, and thus are not intended as a definition of the limits of the present invention, and wherein:

FIG. 1 is a perspective view of the exterior of a radiological imaging apparatus according to an embodiment;

FIG. 2 is a block diagram showing an equivalent circuit of the radiological imaging apparatus;

FIG. 3 is a timing chart for explaining the timing to apply an on-state voltage to each scan line of the radiological imaging apparatus in a case where imaging is performed according to a cooperative method;

FIG. 4 is a timing chart for explaining the timing to apply an on-state voltage to each scan line of the radiological imaging apparatus in a case where imaging is performed according to a non-cooperative method;

FIG. 5 is a timing chart for explaining an offset data readout process to be performed by repeating the same process sequence as the process sequence shown in FIG. 4;

FIG. 6 is a diagram showing a radiological imaging system that includes the radiological imaging apparatus, an irradiation device, and an input unit;

FIG. 7 is a diagram for explaining the timings to switch the imaging mode in the radiological imaging apparatus according to the embodiment, and the timing to perform each process;

FIG. 8 is a diagram showing an example of a bar code shown on an irradiation record;

FIG. 9 is a diagram showing an example of imaging order information; and

FIG. 10 is a diagram showing a radiological imaging system that includes the radiological imaging apparatus and a console.

### DESCRIPTION OF THE PREFERRED EMBODIMENTS

Hereinafter, a radiological imaging apparatus and a radiological imaging system according to an embodiment of the present invention will be described with reference to the drawings. However, the scope of the invention is not limited to the illustrated examples.

In the description below, a radiological imaging apparatus of a so-called indirect type that includes a scintillator and the like, and obtains image data with radiation detecting elements that convert emitted radiation into light of another wavelength such as visible light with the scintillator will be described as a radiological imaging apparatus. However, the present invention can also be applied to a radiological imaging apparatus of a so-called direct type that detects radiation with radiation detecting elements without any scintillator and the like.

[Radiological Imaging Apparatus]

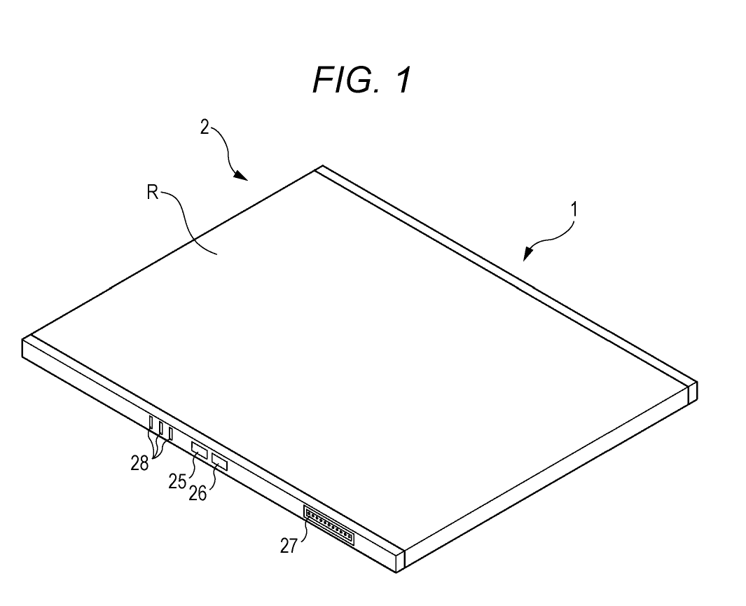

The fundamental structure and the like of a radiological imaging apparatus according to an embodiment are now described. FIG. 1 is a perspective view of the exterior of the radiological imaging apparatus.

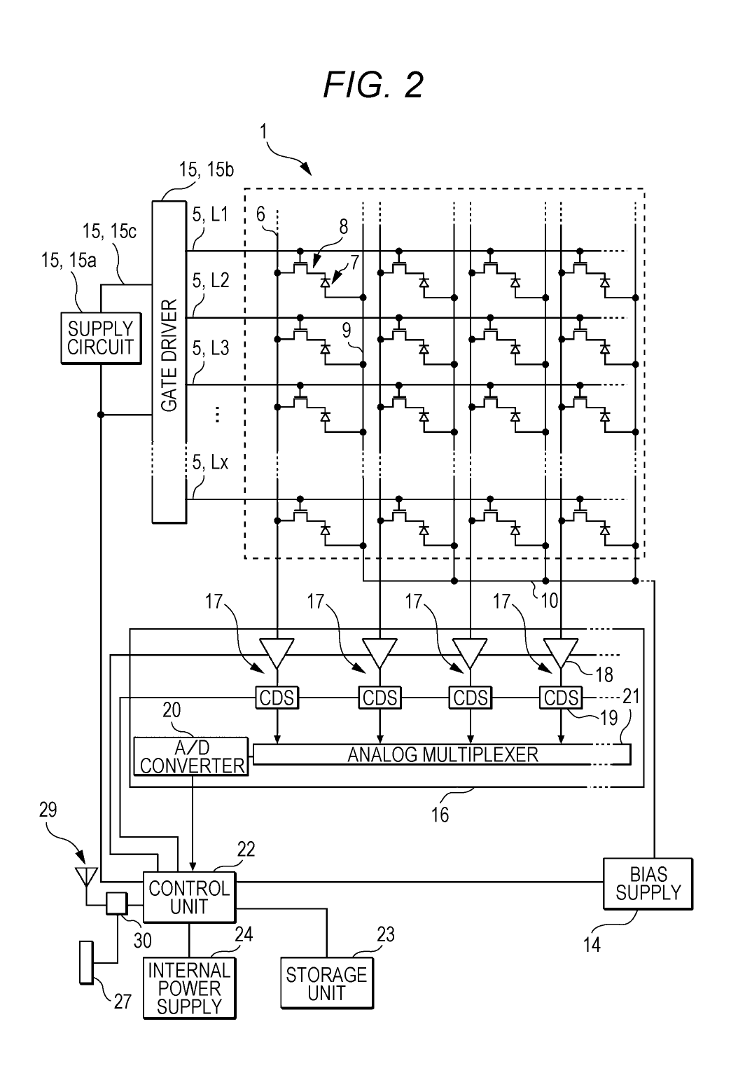

In this embodiment, the radiological imaging apparatus 1 has the later described radiation detecting elements 7 and the like housed in a housing 2. A power switch 25, a select switch 26, a connector 27, indicators 28, and the like are provided on one side surface of the housing 2. Although not shown in the drawing, an antenna 29 (see FIG. 2, which will be later referred to) for communicating with the later described console or the like is provided, for example, on the side surface on the opposite side of the housing 2 in this embodiment. When communicating with the outside in a wireless manner, the radiological imaging apparatus 1 uses the antenna 29. When communicating with the outside in a wired manner, the radiological imaging apparatus 1 has a cable (not shown) or the like connected to the connector 27 to perform communication.

FIG. 2 is a block diagram showing an equivalent circuit of the radiological imaging apparatus. As shown in FIG. 2, in the radiological imaging apparatus 1, radiation detecting elements 7 are arranged two-dimensionally (in a matrix fashion) on a sensor board (not shown). Each of the radiation detecting elements 7 is designed to generate charge in accordance with the dose of radiation to which it is subjected. The respective radiation detecting elements 7 are connected to bias lines 9, and the bias lines 9 are connected to a connecting wire 10. The connecting wire 10 is connected to a bias supply 14, and reverse bias voltage is applied from the bias supply 14 to the respective radiation detecting elements 7 via the bias lines 9 and the like.

Thin-film transistors (hereinafter referred to as TFTs) 8 are connected as switching elements to the respective radiation detecting elements 7, and the TFTs 8 are connected to signal lines 6. In a scanning drive unit 15, a gate driver 15b switches voltage between on-state voltage and off-state voltage supplied from a power supply circuit 15a via an interconnect 15c, and the voltage is applied to respective lines L1 through Lx of scan lines 5. When the on-state voltage is applied to the TFTs 8 via the scan lines 5, each TFT 8 enters an on-state, and releases the charge stored in the corresponding radiation detecting element 7 to the corresponding signal line 6. When the off-state voltage is applied to the TFTs 8 via the scan lines 5, each TFT 8 enters an off-state, cuts off the conduction between the radiation detecting element 7 and the signal line 6, and causes the charge generated in the radiation detecting element 7 to accumulate in the radiation detecting element 7.

A readout IC 16 includes readout circuits 17, and the signal lines 6 are connected to the respective readout circuits 17. In an image data D generation process, charge is released from the radiation detecting elements 7. The charge then flows into the readout circuits 17 via the signal lines 6, and voltage values corresponding to the quantities of charge are output from amplifier circuits 18. Correlated double sampling circuits (shown as “CDS” in FIG. 2) 19 read the image data D, which is the analog values of the voltage values output from the amplifier circuits 18, and outputs the image data D to the downstream side. The output image data D is sequentially transmitted to an A/D converter 20 via an analog multiplexer 21, are sequentially converted into digital image data D by the A/D converter 20, and are sequentially output and stored into a storage unit 23.

A control unit 22 is formed with a computer in which a CPU (Central Processing Unit), a ROM (Read Only Memory), a RAM (Random Access Memory), an input/output interface, and the like are connected by a bus, an FPGA (Field Programmable Gate Array), or the like (not shown). The control unit 22 may be formed with a special-purpose control circuit. The storage unit 23 formed with an SRAM (Static RAM), an SDRAM (Synchronous DRAM), a NAND flash memory, or the like is connected to the control unit 22. Also, a communication unit 30 that communicates with the outside in a wired or wireless manner via the antenna 29 and the connector 27 is connected to the control unit 22.

Further, an internal power supply 24 that supplies necessary power to the respective functional units such as the scanning drive unit 15, the readout circuits 17, the storage unit 23, and the bias supply 14 is connected to the control unit 22. In the image data D generation process, the control unit 22 controls operations of the scanning drive unit 15, the readout circuits 17, and the like in the above described manner, so that charge is read from the respective radiation detecting elements 7, and the image data D is generated.

The radiological imaging apparatus 1 according to this embodiment can be mounted on an imaging stand (not shown), and be then used for imaging. However, the radiological imaging apparatus 1 may not be mounted on an imaging stand, but may be attached directly to the body of a patient as the object, or be inserted between a patient and a bed, for example.

[Power Consuming Mode of the Radiological Imaging Apparatus]

In this embodiment, the control unit 22 is designed to be able to switch the imaging mode of the radiological imaging apparatus 1 between an imaging enabled mode in which the image data D generation process can be performed, and an imaging disabled mode in which the image data D generation process cannot be performed.

In the imaging enabled mode, under the control of the control unit 22, power is supplied from the above described internal power supply 24 to the respective functional units necessary for imaging, such as the scanning drive unit 15, the readout IC 16, and the bias supply 14, so that the image data D generation process can be performed.

The imaging disabled mode may be a power saving mode (also referred to as the sleep mode or the like) in which power is supplied only to the communication unit 30 and the like necessary for receiving signals from outside, or may be a mode such as a standby mode in which power is supplied to the scanning drive unit 15 and the like, but any operation necessary for imaging is disabled so that the image data D generation process cannot be performed. When the imaging mode of the radiological imaging apparatus 1 is the imaging disabled mode, the radiological imaging apparatus 1 does not generate any image data D even if the radiological imaging apparatus 1 is subjected to radiation via an object.

The timing for the control unit 22 to switch the imaging mode of the radiological imaging apparatus 1 between the imaging enabled mode and the imaging disabled mode will be described later in detail.

[Processes to be Performed at Times of Imaging]

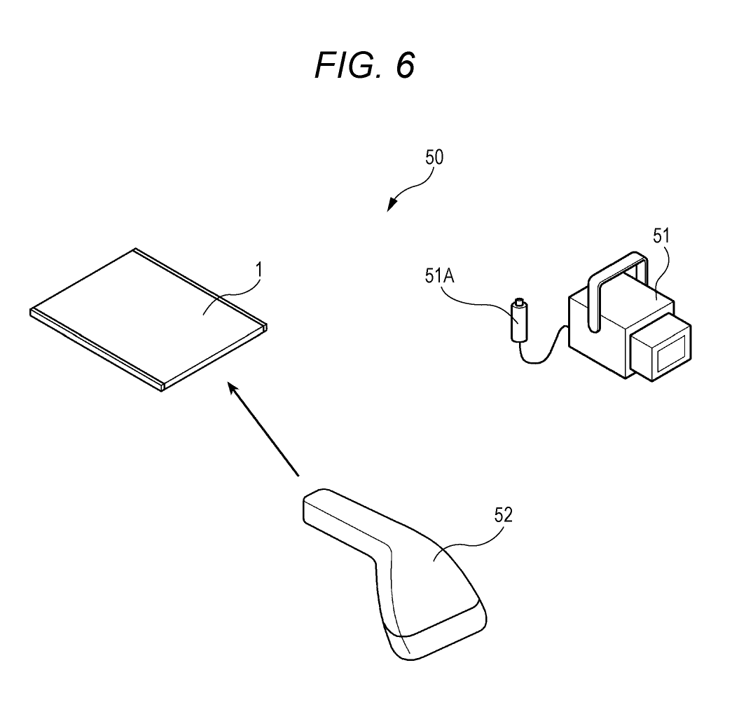

The processes to be performed by the radiological imaging apparatus 1 at times of imaging are now described. There are differences between the process to be performed by the radiological imaging apparatus 1 when imaging is performed while the radiological imaging apparatus 1 and an irradiation device 51 (see FIG. 6, which will be later referred to) cooperate and exchange signals (this imaging method will be hereinafter referred to as the cooperative method), and the process to be performed by the radiological imaging apparatus 1 when imaging is performed while the radiological imaging apparatus 1 and the irradiation device 51 do not exchange any signals (this imaging method will be hereinafter referred to as the non-cooperative method).

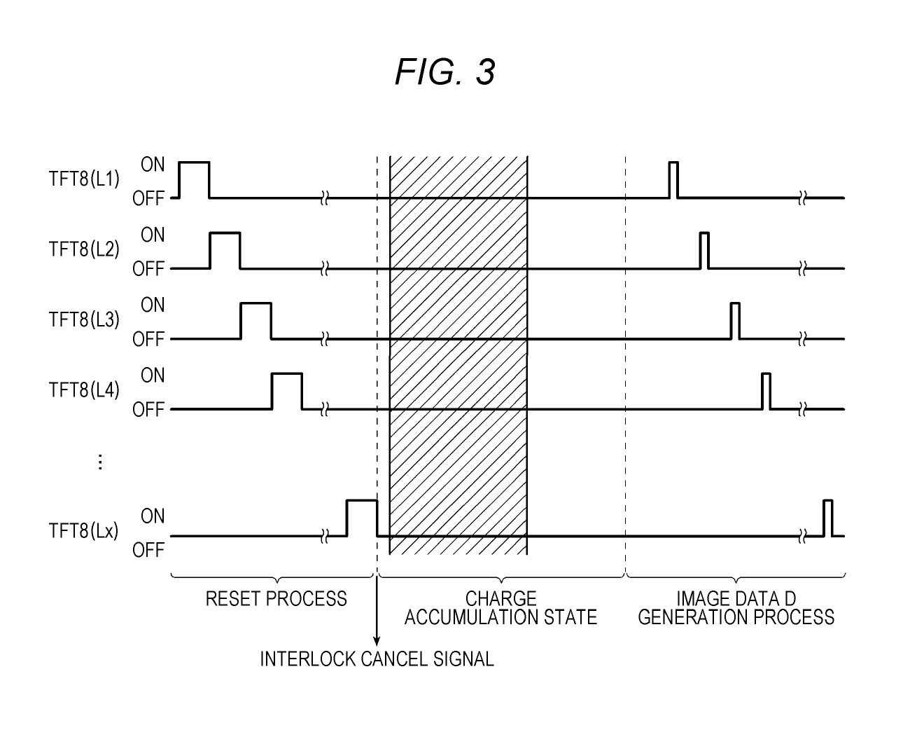

In the case of the cooperative method, the control unit 22 of the radiological imaging apparatus 1 causes the gate driver 15b (see FIG. 2) of the scanning drive unit 15 to sequentially apply the on-state voltage to the respective lines L1 through Lx of the scan lines 5, so that the respective TFTs 8 sequentially enter an on-state, for example, as shown in the left-side portion of FIG. 3. A reset process is performed on the radiation detecting elements 7 by removing the charge remaining in each of the radiation detecting elements 7 in this manner.

When an operator who is a radiological technologist or the like operates (or completely presses) an irradiation switch 51A (see FIG. 6, which will be later referred to) of the irradiation device 51 to emit radiation, an irradiation start signal is transmitted from the irradiation device 51 to the radiological imaging apparatus 1. Receiving the irradiation start signal, the control unit 22 of the radiological imaging apparatus 1 ends the reset process after applying the on-state voltage to the last line Lx of the scan lines 5 and performing the reset process on the corresponding radiation detecting element 7, as shown in FIG. 3. The control unit 22 then applies the off-state voltage from the gate driver 15b to the respective lines L1 through Lx of the scan lines 5, to put the respective TFTs 8 into an off-state. The charge generated in the respective radiation detecting elements 7 by irradiation is stored in the respective radiation detecting elements 7. This state is referred to as the charge accumulation state. At the same time, the control unit 22 of the radiological imaging apparatus 1 transmits an interlock cancel signal to the irradiation device 51.

Receiving the interlock cancel signal, the irradiation device 51 instantly emits radiation. It should be noted that the shaded portion in FIG. 3 indicates the period during which the irradiation device 51 emits radiation. At the time when a predetermined accumulation time τ has passed since the charge accumulation state started, the control unit 22 of the radiological imaging apparatus 1 applies the on-state voltage from the gate driver 15b sequentially to the respective lines L1 through Lx of the scan lines 5, and performs the image data D generation process by reading the image data D from the respective radiation detecting elements 7 in the above described manner, as shown in the right-side portion of FIG. 3.

In the case of the non-cooperative method, on the other hand, no signals are exchanged between the radiological imaging apparatus 1 and the irradiation device 51. In view of this, in the case of the non-cooperative method, the radiological imaging apparatus 1 detects a start of radiation emission from the irradiation device 51. In a case where imaging is performed by the non-cooperative method, the control unit 22 of the radiological imaging apparatus 1 performs a process of detecting a start of radiation emission. Thus, the radiological imaging apparatus 1 can detect a start of radiation emission and accurately perform imaging even in a case where any signals are not exchanged (or cannot be exchanged) between the radiological imaging apparatus 1 and the irradiation device 51.

Examples of methods by which the radiological imaging apparatus 1 can detect radiation emission include the methods disclosed in JP 2009-219538 A, WO 2011/135917 A, and WO 2011/152093 A. As for the details of those methods, see the above publications and the like.

In the example specifically described below, the radiological imaging apparatus 1 is designed to perform processes according to the non-cooperative method, and detect a start of radiation emission in accordance with leak data “dleak” disclosed in the above mentioned WO 2011/135917 A and a value calculated from the leak data.

The leak data “dleak” is the data obtained by applying the off-state voltage from the gate driver 15b to the respective lines L1 through Lx of the scan lines 5, and reading, with the readout circuits 17, the charge leaking from the respective radiation detecting elements 7 to the signal lines 6 via the TFTs 8 in an off-state. When radiation is emitted to the radiological imaging apparatus 1, the value of the read leak data “dleak” becomes larger, and a start of radiation emission can be detected from the increase.

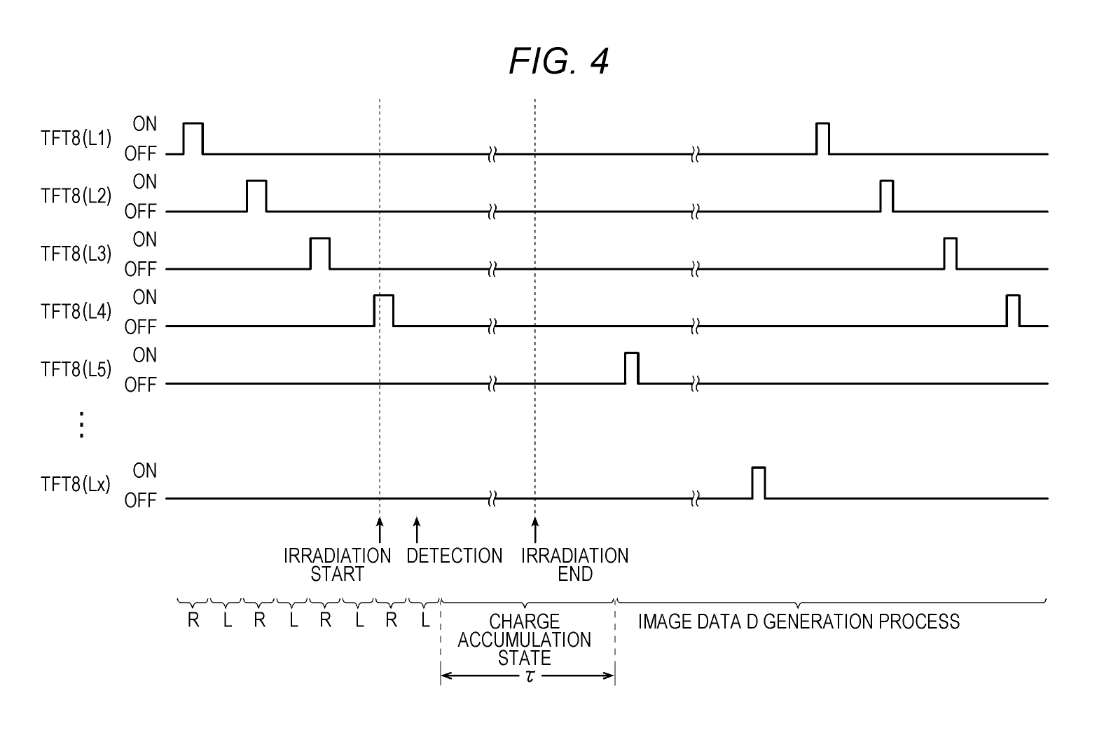

Since the leak data “dleak” is the data that is read when the respective TFTs 8 are in an off-state as described above, dark charge (also called “dark current” or the like) continues to accumulate in the respective radiation detecting elements 7 if the TFTs 8 remain in the off-state. In view of this, the process of reading the leak data “dleak” (this process is indicated by “L” in FIG. 4) and the process of resetting the radiation detecting elements 7 by sequentially applying the on-state voltage from the gate driver 15b to the respective lines L1 through Lx of the scan lines 5 (this process is indicated by “R” in FIG. 4) are alternately performed as shown in the left-side portion of FIG. 4.

When an operator who is a radiological technologist or the like operates the irradiation switch 51A to cause the irradiation device 51 to emit radiation as described above, the leak data “dleak” read in the process of reading the leak data “dleak” at a certain time is conspicuously large, and accordingly, the radiological imaging apparatus 1 can detect a start of radiation emission in accordance with the read leak data “dleak” (see “DETECTION” in FIG. 4).

After detecting a start of radiation emission in the above manner, the control unit 22 of the radiological imaging apparatus 1 applies the off-state voltage from the gate driver 15b to the respective lines L1 through Lx of the scan lines 5, to enter the charge accumulation state. At the time when a predetermined accumulation time τ has passed since the charge accumulation state started, the on-state voltage is applied from the gate driver 15b sequentially to the respective lines L1 through Lx of the scan lines 5, and the image data D generation process is performed by reading the image data D from the respective radiation detecting elements 7 in the above described manner, as shown in the right-side portion of FIG. 4.

In the example case shown in FIG. 4, the image data D generation process is performed by starting the on-voltage application at the scan line 5 (the line L5 of the scan lines 5 in FIG. 4) to which the on-state voltage should be applied immediately after the on-state voltage is applied to the scan line 5 (the line L4 of the scan lines 5 in FIG. 4) to perform the reset process (R) immediately before the leak data “dleak” readout process (L) in which the start of radiation emission has been detected. However, the image data D generation process may be performed by sequentially applying the on-state voltage, starting from the first line L1 of the scan lines 5, for example, as in the case of the cooperative method (see FIG. 3).

[Offset Data Readout Process]

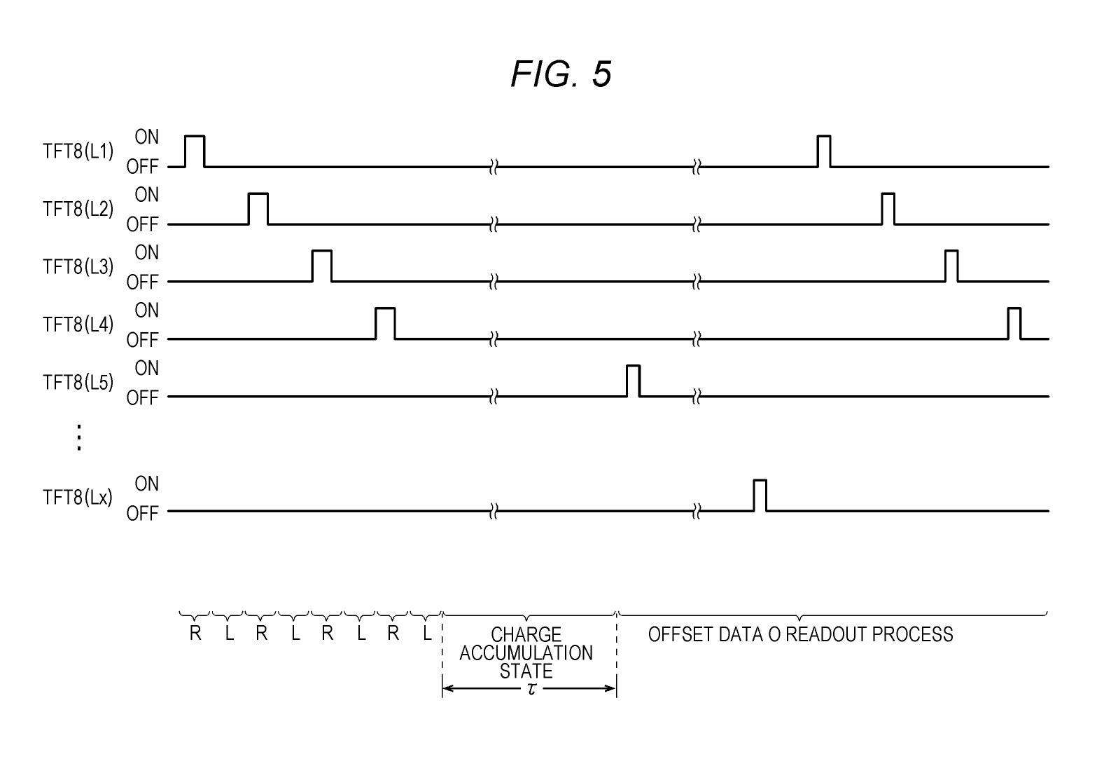

According to both the cooperative method and the non-cooperative method, the control unit 22 of the radiological imaging apparatus 1 performs an offset data O readout process as shown in FIG. 5, for example, after performing the image data D generation process in the above manner. FIG. 5 shows the offset data O readout process to be performed in the case of the non-cooperative method illustrated in FIG. 4.

Specifically, the control unit 22 of the radiological imaging apparatus 1 performs the image data D generation process, for example, as shown in FIG. 4. After performing the reset process on the radiation detecting elements 7 of a predetermined frame, the control unit 22 repeats the same process sequence as the process sequence ending with the image data D generation process shown in FIG. 4, to perform the process of reading the offset data O out of the respective radiation detecting elements 7, as shown in FIG. 5. In the charge accumulation state prior to the offset data O readout process, no radiation is emitted to the radiological imaging apparatus 1.

As shown in FIG. 5, prior to the charge accumulation state in the offset data O readout process, the readout circuits 17 may be made to conduct a readout operation, to perform a leak data “dleak” readout process (L). In this case, there is no need to perform the process of detecting a start of radiation emission, and therefore, the leak data “dleak” readout process (L) may not be performed. In a case where imaging is performed by the cooperative method, the control unit 22 of the radiological imaging apparatus 1 performs the offset data O readout process by repeating the same process sequence as the process sequence shown in FIG. 3 while any radiation is not emitted to the radiological imaging apparatus 1.

After reading the offset data O in the above manner, the control unit 22 of the radiological imaging apparatus 1 stores the offset data O into the storage unit 23 like the image data D.

In the later radiological image generation process, the offset data O is subtracted from the image data D of each radiation detecting element 7 according to the equation (1) shown below, to calculate true image data D* (or the image data that is based on the charge generated in each radiation detecting element 7 by irradiation, and do not contain any noise component such as dark charge).

D*=D−O (1)

In a case where the true image data D* of each radiation detecting element 7 calculated according to the above equation (1) is stored into the storage unit 23 of the radiological imaging apparatus 1, the amount of data to be stored into the storage unit 23 is reduced by half, compared with the amount of data to be stored into the storage unit 23 in a case where the image data D of each radiation detecting element 7 and the offset data O are stored into the storage unit 23. The number of radiological images that can be stored into the storage unit 23 (or the number of times imaging can be performed) can be increased accordingly.

In view of the above, at the time when the offset data O is read out, the control unit 22 of the radiological imaging apparatus 1 can calculate the true image data D* by subtracting the offset data O from the image data D according to the above equation (1) for each radiation detecting element 7, and store the calculated true image data D* into the storage unit 23.

The image data D and the offset data O stored in the storage unit 23 of the radiological imaging apparatus 1, or the true image data D* calculated as above and stored in the storage unit 23 is transmitted later to a console, and a radiological image generation process and the like are performed in accordance with those data in the console. This aspect will be described later in detail.

[Structures of the Radiological Imaging Apparatus and the Like Unique to the Present Invention]

Next, the structures of the radiological imaging apparatus 1 and a radiological imaging system 50 unique to the present invention are described.

This embodiment is based on the assumption that the independent radiological imaging apparatus 1 that performs imaging free of the control of any console, as shown in FIG. 6, and the independent irradiation device 51 are brought to an imaging location (such as a bedroom in a hospital, a patient's home, or a place where racehorses with broken bones are treated), and perform imaging in such a location.

In this case, the obtained image data D and the offset data O, or the true image data D* is accumulated in the storage unit 23 of the radiological imaging apparatus 1. At the time when a series of imaging has been completed, an operator who is a radiological technologist or the like brings the radiological imaging apparatus 1 to the place where a console is set, and the image data D and the offset data O, or the true image data D* is transmitted from the radiological imaging apparatus 1 to the console.

The present invention can be applied not only in an imaging location such as a patient's home not under a suitable imaging environment as described above, but also in a case where the independent radiological imaging apparatus 1 is brought into an imaging room in a hospital or the like under a suitable imaging environment.

In cases where the independent radiological imaging apparatus 1 and the irradiation device 51 are brought to an imaging location and perform imaging, the irradiation device 51 is often unable to exchange signals with the radiological imaging apparatus 1, and therefore, imaging is likely to be performed according to the non-cooperative method. However, in a case where the irradiation device 51 is designed to be able to exchange signals with the radiological imaging apparatus 1, imaging can be performed according to the cooperative method.

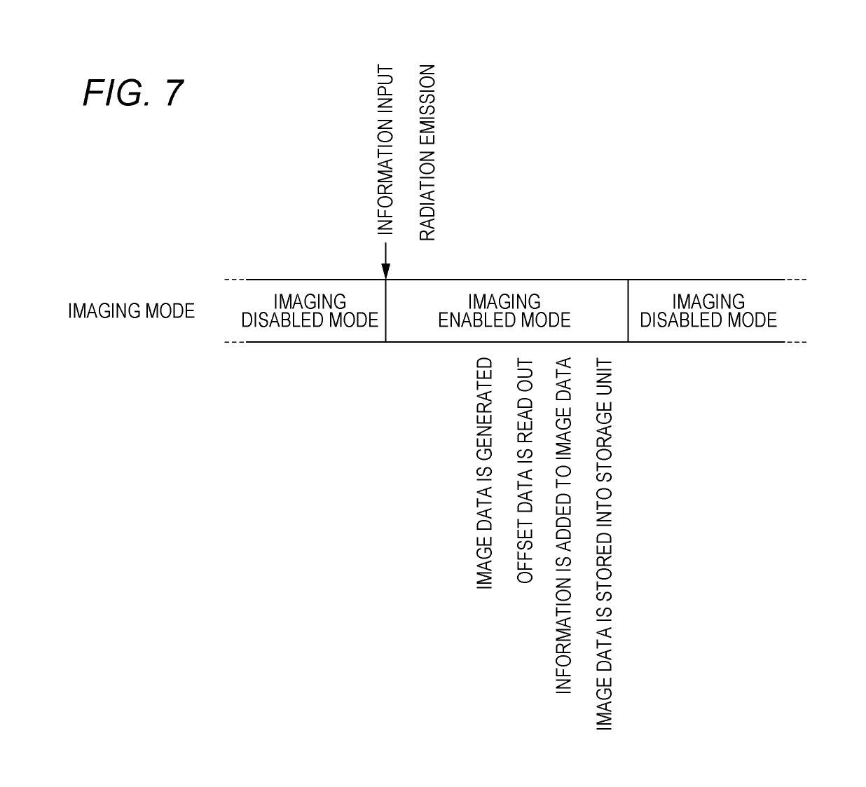

In this embodiment, when at least one kind of information of patient information and imaged site information is input from an input unit 52 (see FIG. 6), the control unit 22 of the radiological imaging apparatus 1 switches the above described imaging mode of the apparatus from the imaging disabled mode to the imaging enabled mode, as shown in FIG. 7. Before any information is input from the input unit 52, the imaging mode is the imaging disabled mode.

After the information is attached to the image data D generated in the above described generation process at the time of imaging performed with radiation emitted in the imaging enabled mode (or to the true image data D* calculated according to the above equation (1): hereinafter, the true image data D* will also be referred to as the image data D), and is stored into the storage unit 23, the imaging mode of the apparatus is switched from the imaging enabled mode to the imaging disabled mode.

This aspect is described below in detail with a specific example structure. For example, when conducting radiological imaging, the operator, who is a radiological technologist or the like, normally carries an irradiation record for recording the imaging conditions (such as the tube voltages, the tube currents, and the irradiation time (or mAs values) set in the irradiation device 51), the imaging situations, and the like at times of imaging.

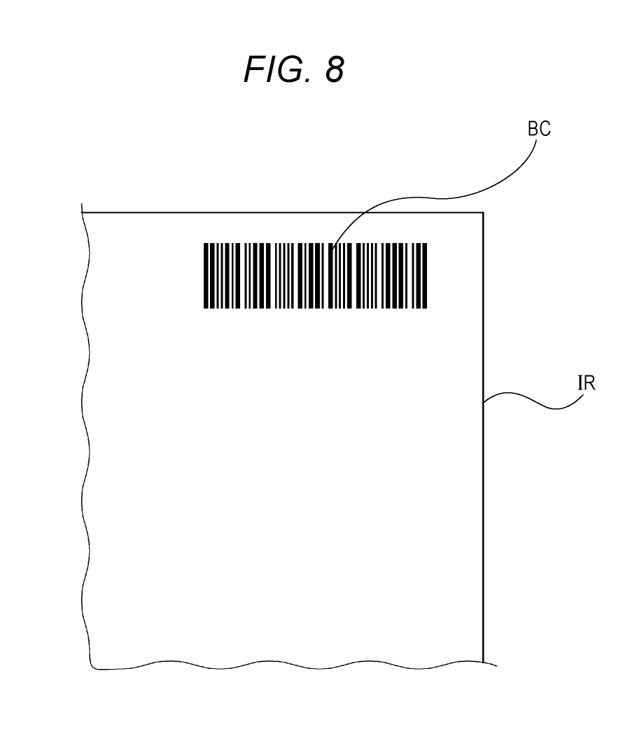

With this structure, a bar code BC in which the patient information such as the name of the patient as the object of the imaging and the patient ID, and the imaged site information indicating the front side of the patient's chest or the like are written is printed beforehand on a paper sheet of an irradiation record IR, for example, as shown in FIG. 8. The bar code BC in the irradiation record IR can be read with a bar code reader, and the patient information and the imaged site information can be transmitted and input to the radiological imaging apparatus 1.

In this case, the bar code reader is equivalent to the input unit 52. The operator, who is a radiological technologist or the like, brings the bar code reader to the imaging location in this case. The patient information and the imaged site information are transmitted and input from the input unit 52 to the radiological imaging apparatus 1 through wireless communication using Bluetooth (a registered trade name), a wireless LAN (Local Area Network), or IrDA (Infrared Data Association), or through cable communication involving a cable (not shown) connecting the input unit 52 and the radiological imaging apparatus 1, for example.

When the patient information and the imaged site information are input as described above, the control unit 22 of the radiological imaging apparatus 1 switches the imaging mode of the apparatus from the imaging disabled mode to the imaging enabled mode. In a case where imaging is performed according to the non-cooperative method, the process of reading the leak data “dleak” (L) and the process of resetting the radiation detecting elements 7 (R) are started, so that the process of detecting a start of radiation emission is started, as shown in FIG. 4. In a case where imaging is performed according to the cooperative method, the process of resetting the radiation detecting elements 7 is started as shown in FIG. 3, so that the apparatus is prepared for the imaging.

After radiation for imaging is emitted to the radiological imaging apparatus 1 from the irradiation device 51 via the object (not shown) as described above, and the image data D generation process (see FIG. 4 and others) and the offset data O readout process (see FIG. 5) are performed, the control unit 22 of the radiological imaging apparatus 1 attaches the input patient information and imaged site information to the generated image data D (or the true image data D* as described above; this also applies in the description below) by writing the information into the header or the like of the image data D.

The control unit 22 of the radiological imaging apparatus 1 then stores the image data D accompanied by the patient information and the like into the storage unit 23. After that, the control unit 22 switches the imaging mode of the apparatus from the imaging enabled mode to the imaging disabled mode. In a case where the image data D and the offset data O are stored into the storage unit 23 separately from each other, the patient information and the like may also be attached to the offset data O.

[Functions]

Next, the functions of the radiological imaging apparatus 1 and the radiological imaging system 50 according to this embodiment are described.

When the power switch 25 (see FIG. 1) is switched on by the operator, who is a radiological technologist or the like, the control unit 22 of the radiological imaging apparatus 1 performs a predetermined initial operation such as the process of resetting the radiation detecting elements 7, and then switches the imaging mode of the apparatus to the imaging disabled mode. If the imaging disabled mode is also the standby mode at that point of time, the control unit 22 controls the scanning drive unit 15, the readout circuits 17 (see FIG. 2), and the like not to operate.

If the imaging disabled mode is the power saving mode, for example, the control unit 22 of the radiological imaging apparatus 1 causes the respective functional units to perform the initial operation as described above, and then controls the internal power supply 24 to supply power only to the necessary functional units such as the communication unit 30. The control unit 22 then enters a sleep state. In this manner, the control unit 22 switches the imaging mode of the apparatus to the imaging disabled mode after the initial operation of each functional unit is completed.

When the imaging mode of the radiological imaging apparatus 1 is the imaging disabled mode, the radiological imaging apparatus 1 does not perform the image data D generation process even if radiation is emitted from the irradiation device 51 to the radiological imaging apparatus 1. That is, even when radiation is emitted to the radiological imaging apparatus 1, the image data D is not generated and stored into the storage unit 23 in the radiological imaging apparatus 1.

The operator, who is a radiological technologist or the like, then reads the bar code printed on or bonded onto a paper sheet of the irradiation record with the bar code reader (or the input unit 52), and the patient information and the imaged site information written in the bar code are transmitted and input to the radiological imaging apparatus 1. If the control unit 22 of the radiological imaging apparatus 1 is in the sleep state at that point of time, the control unit 22 wakes up in accordance with a wake-up signal from the communication unit 30 having received the above information.

At that point of time, the control unit 22 switches the imaging mode of the apparatus from the imaging disabled mode to the imaging enabled mode. Specifically, the control unit 22 causes the internal power supply 24 to supply power to the scanning drive unit 15, the readout circuits 17, and the like, and starts the process of detecting a start of radiation emission if the imaging is to be performed according to the non-cooperative method, as described above. If the imaging is to be performed according to the cooperative method, the control unit 22 starts the process of resetting the radiation detecting elements 7.

When radiation is emitted from the irradiation device 51 to the radiological imaging apparatus 1 via the object because the operator, who is a radiological technologist or the like, operates the irradiation switch 51A, the control unit 22 of the radiological imaging apparatus 1 performs the above described control (see FIGS. 3 and 4) to enter the charge accumulation state, so that the input patient information and imaged site information are attached to the generated image data D, and the image data D is stored into the storage unit 23.

After storing the image data D accompanied by the patient information and the like into the storage unit 23, the control unit 22 of the radiological imaging apparatus 1 switches the imaging mode of the apparatus from the imaging enabled mode to the imaging disabled mode. Unless new patient information and the like are input from the input unit 52 to the radiological imaging apparatus 1 thereafter, no more image data D is generated and stored into the storage unit 23 in the radiological imaging apparatus 1 even if radiation is emitted from the irradiation device 51 to the radiological imaging apparatus 1.

As described above, in the radiological imaging apparatus 1 and the radiological imaging system 50 according to this embodiment, the image data D is not generated in the radiological imaging apparatus 1, unless patient information and the like are input from the input unit 52 to the radiological imaging apparatus 1. Only when patient information and the like are input from the input unit 52 to the radiological imaging apparatus 1, is the image data D generated in the radiological imaging apparatus 1, and are the input patient information and imaged site information attached to the image data D (or the true image data D* as described above; this also applies in the description below).

Specifically, with an input of patient information and imaged site information being a trigger, the radiological imaging apparatus 1 in a state where imaging cannot be performed (the imaging disabled mode) enters a state where imaging can be performed (the imaging enabled mode), and attaches the patient information and the imaged site information to the image data D obtained in that state. The radiological imaging apparatus 1 then returns to the state where imaging cannot be performed (the imaging disabled mode).

In other words, the radiological imaging apparatus 1 in a state where imaging cannot be performed becomes capable of imaging when patient information and imaged site information are input. After attaching the patient information and the imaged site information to the image data D generated immediately after the transition to the imaging enabled state, and storing the image data D, the radiological imaging apparatus 1 returns to the state where imaging cannot be performed. Unless new patient information and the like are input, no more image data D is generated in the radiological imaging apparatus 1 even if radiation is emitted to the radiological imaging apparatus 1.

In this manner, patient information and imaged site information are put into a precise one-to-one correspondence relationship with the image data D obtained in accordance with those sets of information in the radiological imaging apparatus 1 and the radiological imaging system 50 according to this embodiment.

Unless the operator, who is a radiological technologist or the like, reads a bar code in a wrong irradiation record, or a bar code printed on a different irradiation record from the irradiation record related to the current imaging, with a bar code reader, patient information and imaged site information can be accurately associated with and attached to the corresponding image data D.

That is, in this embodiment, human errors in associating at least patient information and imaged site information with image data D occurs only when a wrong bar code is read with the bar code reader. Thus, the possibility of human errors can be made much lower than that in a case where the operator, who is a radiological technologist or the like, associates patient information and imaged site information with image data D by looking at or writing down the numbers displayed on the radiological imaging apparatus as in the conventional cases described earlier.

### Effects

As described above, in the radiological imaging apparatus 1 and the radiological imaging system 50 according to this embodiment, even when the radiological imaging apparatus 1 performs imaging free of the control of any console or the like, the possibility of human errors can be dramatically lowered, and patient information and imaged site information can be accurately attached to the corresponding image data D (or the true image data D*; this also applies in the description below).

With this, the imaging order information related to the current imaging can be accurately identified in accordance with the patient information and the imaged site information attached to the corresponding image data D, and the imaging order information can be accurately associated with (or linked to) the radiological image generated in accordance with the image data D or the like. Thus, the following errors can be prevented: a radiological image is associated with wrong imaging order information, and a patient is misdiagnosed with an injury or a disease the patient is not inflicted with, or a patient is misdiagnosed as having recovered from an injury or a disease the patient is still suffering from.

After recognized and confirmed by the operator, who is a radiological technologist or the like, the radiological image accurately associated with the imaging order information is transmitted to a predetermined external system such as a PACS (Picture Archiving and Communication System).

The imaging order information is obtained from an HIS (Hospital Information System) or an RIS (Radiology Information System), or is read from a paper sheet on which the imaging order information is written, or is input directly to a console C. In this manner, the console C obtains the imaging order information.

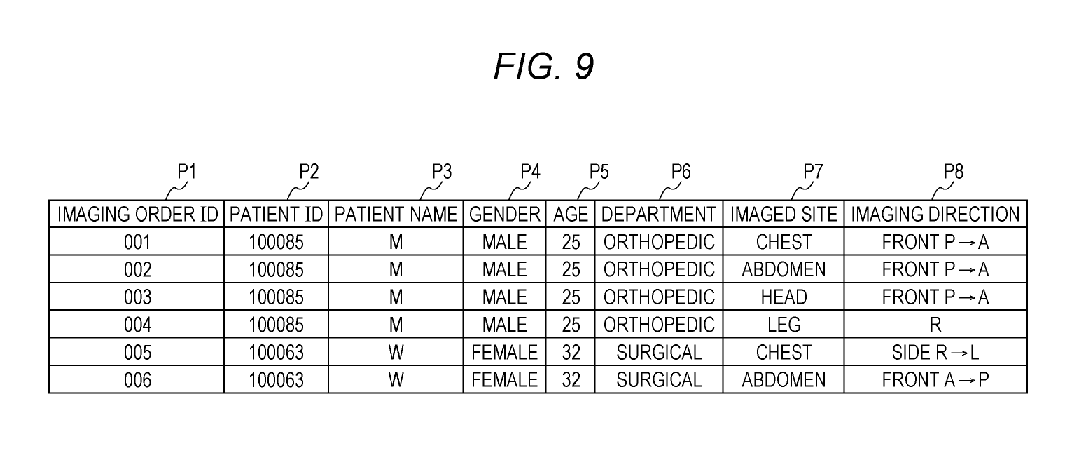

As shown in FIG. 9, for example, the imaging order information includes “patient ID” P2, “patient name” P3, “gender” P4, “age” P5, and “department” P6, which constitute the patient information, and “imaged site” P7 and “imaging direction” P8, which constitute the imaged site information. “Imaging order ID” P1 is automatically assigned to each set of imaging order information in accordance with the sequence of acceptance of imaging orders.

In accordance with the imaging order information, an irradiation record is normally created for each set of imaging order information. When an irradiation record is created, the patient information and the imaged site information designated in the imaging order information are written into a bar code that is then printed on the irradiation record, or a printed bar code is bonded onto the irradiation record, so that the bar code is shown on the irradiation record. In this manner, the patient information and the imaged site information that are input to the radiological imaging apparatus 1 via the input unit 52 and are then attached to the image data D can be in the same format as the patient information and the imaged site information in the imaging order information. The imaging order ID can also be input to the radiological imaging apparatus 1 and be attached to the image data D.

With this, the operator, who is a radiological technologist or the like, can relatively easily identify the imaging order information containing the patient information and the imaged site information corresponding to the patient information and the imaged site information attached to the image data D, and can accurately associate the imaging order information with the radiological image generated in accordance with the image data D or the like.

### First Modification

In the above described embodiment, the operator, who is a radiological technologist or the like, identifies the imaging order information related to the current imaging in accordance with the patient information and the imaged site information attached to the image data D or the true image data D*, and associates the imaging order information with the corresponding radiological image in a console C. However, the console C that generates the radiological image in accordance with the image data D or the like as described above may be designed to associate the imaging order information with the radiological image.

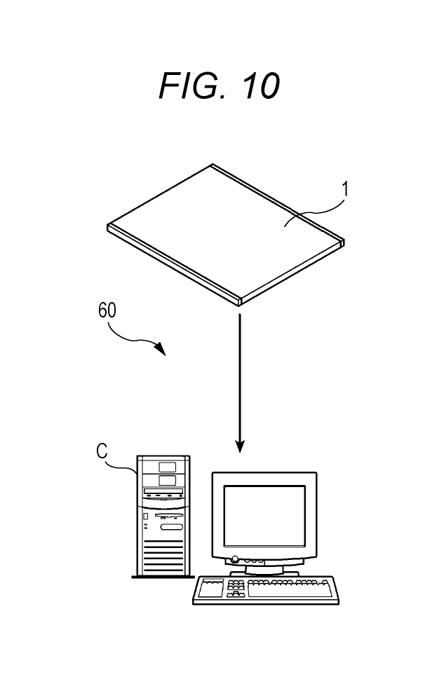

A radiological imaging system 60 in this case includes a radiological imaging apparatus 1 and the console C, as shown in FIG. 10. As described above, when image data D and offset data O, or true image data D* is transmitted from the radiological imaging apparatus 1, the console C generates a radiological image by performing image processing on the true image data D* calculated in accordance with the transmitted image data D and offset data O, or on the transmitted true image data D*. The image processing includes gain correction, defect correction, and gradation processing in accordance with the imaged site.

The console C can associate the generated radiological image with the imaging order information corresponding to the patient information and the imaged site information attached to the image data D or the true image data D* on which the radiological image is based.

With this structure, the generated radiological image can be accurately associated with the corresponding imaging order information in accordance with the patient information and the imaged site information as described above. Furthermore, the console C automatically carries out the associating procedure, and the operator, who is a radiological technologist or the like, only has to check whether the association is correct. Thus, the process of associating a radiological image with the corresponding imaging order information becomes much easier.

[Sequence of Data Transmission from the Radiological Imaging Apparatus to the Console]

In both the above described embodiment (or a case where the operator, who is a radiological technologist or the like, associates a radiological image with the corresponding imaging order information) and the first modification (or a case where the console C carries out the associating procedure), the patient information and the imaged site information are accurately attached to the image data D or the true image data D* according to the present invention, as described above.

When the image data D or the true image data D* is transmitted from the radiological imaging apparatus 1 to the console C, radiological images can be accurately associated with the imaging order information in accordance with the patient information and the imaged site information attached to the image data D or the true image data D*, even if the transmission is not performed in the same sequence as the imaging sequence. In view of this, the image data D or the true image data D* may be transmitted from the radiological imaging apparatus 1 to the console C in the same sequence as the imaging sequence, but does not need to be transmitted in the same sequence as the imaging sequence. The sequence of transmitting the image data D or the true image data D* from the radiological imaging apparatus 1 to the console C can be determined as appropriate.

### Second Modification

In the above described example structure, a bar code shown on an irradiation record is read with a bar code reader serving as the input unit 52. However, a two-dimensional code, such as a QR code (a registered trade name), may be shown on the irradiation record, and the two-dimensional code may be imaged with an imaging device, such as a digital camera, a smartphone, or a tablet terminal. The patient information and the imaged site information written in the two-dimensional code may be then transmitted and input to the radiological imaging apparatus 1. In this case, the imaging device, such as a digital camera, a smartphone, or a tablet terminal, serves as the input unit 52.

The operator, who is a radiological technologist or the like, may input patient information and site information to a smartphone, a tablet, or the like, and then transmit the patient information and the site information to the radiological imaging apparatus 1. In a case where no bar code reader is provided, and the imaging device does not have any function to read patient information and imaged site information from a two-dimensional code, a bar code or a two-dimensional code may be imaged in a photograph or a moving image with an imaging device, for example, and the data of the photograph or the moving image may be attached as the patient information and the imaged site information to image data D or true image data D*.

Instead of imaging a bar code or a two-dimensional code with an imaging device such as a digital camera, a smartphone, or a tablet terminal, it is possible to image a patient or an imaged site or an imaging scene in a photograph and a moving image, and attach the photograph or the moving image as the patient information and the imaged site information to the image data D or the true image data D*. Also, it is possible to input biological information about a fingerprint, the iris, the retina, or a vein as the patient information, or read and input a bar code or the like printed on a patient identification wristband the patient is wearing on his/her wrist. In a case where imaging is performed at a patient's home, it is possible to input location information obtained with a GPS (Global Positioning System) or a gyro sensor, as the patient information.

Alternatively, the radiological imaging apparatus 1 may be designed to have a function to image a bar code or a two-dimensional code, a function to image a patient or an imaged site in a photograph or a moving image, or a function to recognize biological information or location information as the patient information. That is, the radiological imaging apparatus 1 may also be designed to serve as the input unit 52.

### Third Modification

When patient information and imaged site information are input from the input unit 52, the control unit 22 of the radiological imaging apparatus 1 automatically switches the imaging mode of the apparatus from the imaging disabled mode to the imaging enabled mode, as described above. After the patient information and the imaged site information are attached to generated image data D or calculated true image data D*, and are stored into the storage unit 23, the control unit 22 automatically switches the imaging mode of the apparatus from the imaging enabled mode to the imaging disabled mode. As a result, the operator, who is a radiological technologist or the like, might become unable to know which mode is the current imaging mode of the radiological imaging apparatus 1.

In view of this, the control unit 22 of the radiological imaging apparatus 1 may cause the indicators 28 (see FIG. 1) to emit light or blink in a different color for each imaging mode. Alternatively, the control unit 22 may transmit a signal to a smartphone or a tablet terminal being carried by the operator, who is a radiological technologist or the like, every time imaging modes are switched, and cause the smartphone or the tablet terminal to display the current imaging mode of the radiological imaging apparatus 1 on its screen. Also, the radiological imaging apparatus 1, a smartphone, a tablet terminal, or the like may be made to generate sound every time imaging modes are switched, and the operator, which is a radiological technologist or the like, may be notified that imaging modes have been switched.

### Fourth Modification

As described above, in some cases, patient information and imaged site information might be input from the input unit 52 to the radiological imaging apparatus 1 without any radiation emitted to the radiological imaging apparatus 1 (or without any image data D generated) after the previous input of patient information and imaged site information read from a bar code or the like to the radiological imaging apparatus 1 from the input unit 52. In such a case, the operator, who is a radiological technologist or the like, might have input wrong patient information and the like, and therefore, have input the correct patient information and the like. However, the operator might have forgotten to conduct imaging based on the patient information and the like that was input first, and have then input the patient information and the like for the next imaging.

In view of the above, when there are consecutive inputs of patient information and imaged site information from the input unit 52 though the image data D generation process has not been performed, the control unit 22 of the radiological imaging apparatus 1 may cause the indicators 28 to emit light in a predetermined color or blink in a predetermined manner, or generate sound. Alternatively, the control unit 22 may transmit a signal to a smartphone or a tablet terminal being carried by the operator, who is a radiological technologist or the like, and cause the smartphone or the tablet terminal to display a warning on its screen or generate sound, to notify the operator that there have been consecutive inputs of patient information and imaged site information from the input unit 52.

With this structure, it is possible to accurately notify the operator, who is a radiological technologist or the like, that there have been consecutive inputs of patient information and imaged site information from the input unit 52, though the image data D generation process has not been performed.

In this case, at the time of issuing a warning, the control unit 22 of the radiological imaging apparatus 1 may automatically switch the imaging mode of the apparatus to the imaging disabled mode, and cause the input unit 52 to start again from inputting patient information and imaged site information. Alternatively, despite issuing a warning, the control unit 22 may determine that the operator, who is a radiological technologist or the like, have input the correct patient information and the like, and maintains the apparatus in the imaging enabled mode. The control unit 22 then deletes the previously input patient information and the like, and keeps the last input patient information and the like. The control unit 22 then performs processing, regarding the last input patient information and the like as the valid patient information and the like.

As described above, in a case where there are consecutive inputs of patient information and imaged site information from the input unit 52, it is possible to determine into which state the radiological imaging apparatus 1 is to be put, and the operator, who is a radiological technologist or the like, is notified of the decision. Alternatively, the operator, who is a radiological technologist or the like, can determine into which state the radiological imaging apparatus 1 is to be put in a case where patient information and imaged site information are consecutively input from the input unit 52 (or the operator can customize the radiological imaging apparatus 1).

### Fifth Modification

As described above, there are cases where the irradiation start signal is not transmitted from the irradiation device 51 even when a predetermined time has passed since the patient information and the imaged site information were input from the input unit 52 to the radiological imaging apparatus 1 (the cooperative method). There also are cases where the radiological imaging apparatus 1 does not detect a start of radiation emission (the non-cooperative method). Further, there are cases where the operator, who is a radiological technologist or the like, operates the select switch 26 (or a cancel button, see FIG. 1) of the radiological imaging apparatus 1. In any of those cases, the control unit 22 of the radiological imaging apparatus 1 can switch the imaging mode of the apparatus from the imaging enabled mode to the imaging disabled mode.

When switching the imaging mode of the apparatus from the imaging disabled mode to the imaging enabled mode, the control unit 22 of the radiological imaging apparatus 1 performs the process of resetting the radiation detecting elements 7 (the cooperative method) or performs the process of detecting a start of radiation emission (the non-cooperative method), as described above. However, if the process is continued though imaging is not performed (or radiation is not emitted from the irradiation device 51), problems such as unnecessary consumption of power supplied from the internal power supply 24 occur in the radiological imaging apparatus 1.

In view of this, the imaging mode of the apparatus is switched from the imaging enabled mode to the imaging disabled mode in the above structure, so that the imaging mode can be automatically switched to the imaging disabled mode when imaging is not performed. Thus, problems such as unnecessary consumption of power supplied from the internal power supply 24 can be appropriately prevented in the radiological imaging apparatus 1.

### Sixth Modification

There are cases where the number of sets of image data D and offset data O or true image data D* stored in the storage unit 23 of the radiological imaging apparatus 1 has reached the upper limit number of data sets that can be stored in the storage unit 23, and the storage unit 23 cannot store anymore image data D or the like. In such a case, image data D or the like accompanied by patient information and imaged site information cannot be stored into the storage unit 23, even if the patient information and the imaged site information are input from the input unit 52 to the radiological imaging apparatus 1.

In view of this, when the amount of the image data D or the like stored in the storage unit 23 has reached the upper limit, the control unit 22 of the radiological imaging apparatus 1 does not accept any more inputs of information such as patient information from the input unit 52, and does not switch the imaging mode of the radiological imaging apparatus 1 from the imaging disabled mode to the imaging enabled mode, so that the radiological imaging apparatus 1 remains in the imaging disabled mode.

Unless such a structure is employed, the storage unit 23 is overwritten with the image data D or the like generated by imaging performed after the amount of the image data D or the like stored in the storage unit 23 has reached the upper limit. As a result, the necessary image data D or the like already stored in the storage unit 23 might be lost. However, with the above described structure, such a problem can be appropriately avoided, and loss of the necessary image data D or the like stored in the storage unit 23 due to overwriting can be prevented without fail.

In such a case, the indicators 28 of the radiological imaging apparatus 1 may be made to emit light in a predetermined color or blink in a predetermined manner, or generate sound. Alternatively, a signal may be transmitted to a smartphone or a tablet terminal being carried by the operator, who is a radiological technologist or the like, and the smartphone or the tablet terminal may be made to display a warning on its screen or generate sound, to notify the operator that the number of data sets stored in the storage unit 23 of the radiological imaging apparatus 1 has reached the upper limit, and that no more inputs from the input unit 52 are to be accepted.

With this structure, the operator, who is a radiological technologist or the like, can be prevented from continuing to conduct imaging without noticing that the storage unit 23 of the radiological imaging apparatus 1 cannot store any more image data D or the like. Thus, the operator can suspend the imaging, replace the radiological imaging apparatus 1 being used with another radiological imaging apparatus 1, or transmit the image data D or the like stored in the storage unit 23 of the radiological imaging apparatus 1 to the console C and store other image data D or the like into the storage unit 23. In this manner, the operator, who is a radiological technologist or the like, can promptly cope with the situation.

### Seventh Modification

In the above described embodiment and modifications, the information that is input from the input unit 52 to the radiological imaging apparatus 1 includes patient information and imaged site information. However, the information that is input from the input unit 52 to the radiological imaging apparatus 1 does not necessarily include both patient information and imaged site information.

For example, in a case where the radiological imaging apparatus 1, the irradiation device 51, and the input unit 52, which are the radiological imaging system 50 shown in FIG. 6, are brought into a bedroom of a hospital or the home of a patient, and imaging is performed on different imaged sites of the same patient, the object of imaging is only the patient, and sets of image data D can be distinguished from one another, as long as the imaged site information is attached to the image data D or the true image data D*.

In a case where the image data D or the like is transmitted from the radiological imaging apparatus 1 to the console C (see FIG. 10), and a radiological image generated in accordance with the image data D or the like is associated with imaging order information in the console C, the imaging order information to be associated with the radiological image can be accurately identified among the sets of imaging order information related to the patient, in accordance with the imaged site information attached to the image data D or the like. Thus, each radiological image can be accurately associated with imaging order information.

In view of the above, when imaging is performed on different imaged sites of the same patient, the information to be attached to the generated image data D or the like may be only the imaged site information. In this case, patient information may also be attached to the image data D or the like, but there is no need to attach the patient information to the image data D or the like.

In such a case, it is assumed that imaging is performed on different imaged sites of the same patient without any intermission. In view of this, maintaining the imaging enabled mode as the imaging mode of the radiological imaging apparatus 1 is preferable to switching the imaging mode to the imaging disabled mode every time imaging is completed (or every time the image data D or the like accompanied by generated information is stored into the storage unit 23) as in the above described embodiment (see FIG. 7), because imaging can be successively performed in the imaging enabled mode.

When an instruction for imaging of different sites of the same patient is issued, the control unit 22 of the radiological imaging apparatus 1 receives an input of information from the input unit 52, and switches the imaging mode of the radiological imaging apparatus 1 from the imaging disabled mode to the imaging enabled mode. After that, the control unit 22 does not switch the imaging mode from the imaging enabled mode to the imaging disabled mode, but maintains the imaging enabled mode. Also, every time image data D is generated as a result of imaging performed in the imaging enabled mode, or every time true image data D* is calculated, the imaged site information that has been input from the input unit 52 immediately before the image data D is generated can be attached to the image data D or the true image data D*, and be stored into the storage unit 23.

Specifically, when an instruction for imaging of different sites of the same patient is issued, the control unit 22 of the radiological imaging apparatus 1 first receives an input of imaged site information from the input unit 52, and switches the imaging mode of the radiological imaging apparatus 1 from the imaging disabled mode to the imaging enabled mode. After that, the control unit 22 maintains the imaging enabled mode as the imaging mode.

That is, the control unit 22 attaches the imaged site information to the image data D or the like and stores the imaged site information into the storage unit 23. However, the control unit 22 does not switch the imaging mode back to the imaging disabled mode. Thereafter, the control unit 22 alternates between receiving an input of imaged site information from the input unit 52 and storing image data D or the like accompanied by the imaged site information into the storage unit 23.

With this structure, imaging can be successively performed on different imaged sites without any intermission in a case where imaging is performed on different imaged sites of the same patient. Also, the image data D or the true image data D* can be accurately associated with the corresponding imaged site information, and the radiological image generated in accordance with the image data D or the like can be accurately associated with imaging order information in accordance with the imaged site information attached to the image data D or the like.

In this case, the number of times imaging is to be consecutively performed may be input to the radiological imaging apparatus 1 in advance or at the time when an input of imaged site information from the input unit 52 is first received. Immediately after the image data D or the true image data D* equivalent to the input number of times of imaging is generated, and the image data D or the true image data D* is stored into the storage unit 23, the control unit 22 of the radiological imaging apparatus 1 may return to the state in the above described embodiment (or switch the imaging mode of the radiological imaging apparatus 1 from the imaging enabled mode to the imaging disabled mode at the time when the last image data D or the like is stored into the storage unit 23).

To transmit the instruction for imaging of different sites of the same patient to the control unit 22 of the radiological imaging apparatus 1, the operator, who is a radiological technologist or the like, may operate the select switch 26 (see FIG. 1) of the radiological imaging apparatus 1 in a predetermined manner, or read a bar code or a two-dimensional code indicating the contents of the instruction with the input unit 52, which is a bar code reader or a smartphone, for example, and then transmit a signal indicating the contents of the instruction to the radiological imaging apparatus 1. Any of these methods can be used.

Furthermore, after the imaging mode of the radiological imaging apparatus 1 is switched from the imaging disabled mode to the imaging enabled mode in the above case, the radiological imaging apparatus 1 remains in the imaging enabled mode. With this, imaging can be successively performed on different imaged sites, without any inputs of imaged site information from the input unit 52. As a result, the operator, who is a radiological technologist or the like, might conduct imaging while forgetting to input imaged site information.

In view of this, in a case where the imaging enabled mode is maintained as the imaging mode while imaging is performed on the same patient, as described above, the control unit 22 of the radiological imaging apparatus 1 can warn that imaging has been continued without any inputs of imaged site information from the input unit 52 (or that image data D or true image data D* is generated or calculated twice or more in a row, without any inputs of imaged site information from the input unit 52).

### Eighth Modification

In a case where the radiological imaging system 50 shown in FIG. 6 is brought to an elementary school or the like, and the front chests of students are successively imaged in mass examination, the imaging is performed only on the front chests, and there are various patients as the objects of imaging (the objects being the students). In this case, sets of image data D or the like can be distinguished from one another, as long as patient information is attached to the image data D or the true image data D*.

In a case where the image data D or the like is transmitted from the radiological imaging apparatus 1 to the console C (see FIG. 10), and a radiological image generated in accordance with the image data D or the like is associated with imaging order information in the console C, the imaging order information to be associated with the radiological image can be accurately identified in accordance with the patient information attached to the image data D or the like. Thus, each radiological image can be accurately associated with imaging order information.

In view of the above, in a case where imaging is performed only once on each patient or where imaging performed on the same imaged sites of different patients, the information to be attached to the generated image data D or the like may be only the patient information. In this case, imaged site information may also be attached to the image data D or the like, but there is no need to attach the imaged site information to the image data D or the like.

In such a case, imaging is likely to be performed successively on patients. In view of this, maintaining the imaging enabled mode as the imaging mode of the radiological imaging apparatus 1 is preferable to switching the imaging mode to the imaging disabled mode every time imaging is completed (or every time the image data D or the like accompanied by generated information is stored into the storage unit 23) as in the above described embodiment, because imaging can be successively performed in the imaging enabled mode.

When an instruction for one-time imaging of different patients is issued, the control unit 22 of the radiological imaging apparatus 1 receives an input of information from the input unit 52, and switches the imaging mode of the radiological imaging apparatus 1 from the imaging disabled mode to the imaging enabled mode. After that, the control unit 22 does not switch the imaging mode from the imaging enabled mode to the imaging disabled mode, but maintains the imaging enabled mode. Also, every time image data D is generated as a result of imaging performed in the imaging enabled mode, or every time true image data D* is calculated, the patient information that has been input from the input unit 52 immediately before the image data D is generated can be attached to the image data D or the true image data D*, and be stored into the storage unit 23.

Specifically, when an instruction for one-time imaging of different patients is issued, the control unit 22 of the radiological imaging apparatus 1 first receives an input of patient information from the input unit 52, and switches the imaging mode of the radiological imaging apparatus 1 from the imaging disabled mode to the imaging enabled mode. After that, the control unit 22 maintains the imaging enabled mode as the imaging mode.

That is, the control unit 22 attaches the patient information to the image data D or the like and stores the patient information into the storage unit 23. However, the control unit 22 does not switch the imaging mode back to the imaging disabled mode. Thereafter, the control unit 22 alternates between receiving an input of patient information from the input unit 52 and storing image data D or the like accompanied by the patient information into the storage unit 23.

With this structure, imaging can be successively performed on different patients without any intermission in a case where imaging is performed only once on each patient or where imaging is performed on the same imaged sites of different patients. Also, the image data D or the true image data D* can be accurately associated with the corresponding patient information, and the radiological image generated in accordance with the image data D or the like can be accurately associated with imaging order information in accordance with the patient information attached to the image data D or the like.

In this case, the number of times imaging is to be consecutively performed may also be input to the radiological imaging apparatus 1 in advance or at the time when an input of patient information from the input unit 52 is first received. Immediately after the image data D or the true image data D* equivalent to the input number of times of imaging is generated, and the image data D or the true image data D* is stored into the storage unit 23, the control unit 22 of the radiological imaging apparatus 1 may return to the state in the above described embodiment (or switch the imaging mode of the radiological imaging apparatus 1 from the imaging enabled mode to the imaging disabled mode at the time when the last image data D or the like is stored into the storage unit 23).

To transmit the instruction for one-time imaging of different patients to the control unit 22 of the radiological imaging apparatus 1, the operator, who is a radiological technologist or the like, may operate the select switch 26 (see FIG. 1) of the radiological imaging apparatus 1 in a predetermined manner, or read a bar code or a two-dimensional code indicating the contents of the instruction with the input unit 52, which is a bar code reader or a smartphone, for example, and then transmit a signal indicating the contents of the instruction to the radiological imaging apparatus 1, as in the above described seventh modification. Any of these methods can be used.

Furthermore, after the imaging mode of the radiological imaging apparatus 1 is switched from the imaging disabled mode to the imaging enabled mode in the eighth modification, the radiological imaging apparatus 1 also remains in the imaging enabled mode. With this, imaging can be successively performed on different patients, without any inputs of patient information from the input unit 52. As a result, the operator, who is a radiological technologist or the like, might conduct imaging while forgetting to input patient information.

In view of this, in a case where the imaging enabled mode is maintained as the imaging mode while imaging is performed on different patients but only once on each patient, as described above, the control unit 22 of the radiological imaging apparatus 1 can warn that imaging has been continued without any inputs of patient information from the input unit 52 (or that image data D or true image data D* is generated or calculated twice or more in a row, without any inputs of patient information from the input unit 52). The eighth modification can also be applied in a case where the shapes of the teeth of laid bodies are imaged for identification, for example, in a scene of a disaster.

### Ninth Modification

In the above described embodiment and modifications, the radiological imaging apparatus 1 is an independent radiological imaging apparatus that performs imaging free of the control of a console. However, the radiological imaging apparatus 1 can be designed to be able to switch its mode between a mode for performing imaging free of the control of a console (or an independent mode) and a mode for performing imaging under the control of a console, for example.

It should be understood that the present invention is not limited to the above described embodiment and modifications, and various changes may be made to them without departing from the scope of the invention.

Although the present invention has been described and illustrated in detail, it is clearly understood that the same is by way of illustrated and example only and is not to be taken by way of limitation, the scope of the present invention being interpreted by terms of the appended claims.

What is claimed is:

1. A radiological imaging apparatus comprising:
a plurality of radiation detecting elements two-dimensionally arranged and configured to generate charge in accordance with a dose of radiation emitted;
a control unit configured to conduct control to perform an image data generation process to generate image data by reading the charge from each of the radiation detecting elements; and
a storage unit configured to store the image data,
wherein the control unit is capable of switching an imaging mode between an imaging enabled mode in which the image data generation process is enabled and an imaging disabled mode in which the image data generation process is disabled,
when at least one kind of information of patient information and imaged site information is input, the control unit switches the imaging mode from the imaging disabled mode to the imaging enabled mode, and
after attaching the information to the image data generated by imaging performed in the imaging enabled mode and storing the image data into the storage unit, the control unit switches the imaging mode from the imaging enabled mode to the imaging disabled mode.

2. The radiological imaging apparatus according to claim 1, wherein, when imaging is performed by a non-cooperative method, the control unit receives an input of the information, switches the imaging mode from the imaging disabled mode to the imaging enabled mode, and starts a process of detecting a start of radiation emission.

3. The radiological imaging apparatus according to claim 1, wherein, when the information is consecutively input though the image data generation process has not been performed, the control unit warns that the information has been consecutively input.

4. The radiological imaging apparatus according to claim 1, wherein, when the number of sets of the image data stored in the storage unit has reached an upper limit number of data sets storable in the storage unit, the control unit does not accept any more inputs of the information, and maintains the imaging disabled mode as the imaging mode.

5. The radiological imaging apparatus according to claim 1, wherein
when an instruction for performing imaging on different imaged sites of the same patient is issued,
the control unit receives an input of the information, switches the imaging mode from the imaging disabled mode to the imaging enabled mode, and maintains the imaging enabled mode, instead of switching the imaging mode from the imaging enabled mode back to the imaging disabled mode, and
every time the image data is generated by imaging performed in the imaging enabled mode, the control unit attaches the imaged site information to the image data, and stores the image data into the storage unit, the imaged site information having been input immediately before the image data is generated.

6. The radiological imaging apparatus according to claim 1, wherein
when an instruction for performing imaging once on each of different patients is issued,
the control unit receives an input of the information, switches the imaging mode from the imaging disabled mode to the imaging enabled mode, and maintains the imaging enabled mode, instead of switching the imaging mode from the imaging enabled mode back to the imaging disabled mode, and
every time the image data is generated by imaging performed in the imaging enabled mode, the control unit attaches the patient information to the image data, and stores the image data into the storage unit, the patient information having been input immediately before the image data is generated.

7. A radiological imaging system comprising:
the radiological imaging apparatus according to claim 1;
an input unit configured to input the at least one kind of information of the patient information and the imaged site information to the radiological imaging apparatus; and
an irradiation device configured to emit radiation to the radiological imaging apparatus.

8. The radiological imaging system according to claim 7, wherein
the information is written in a bar code or a two-dimensional code shown on an irradiation record, and
the input unit is capable of reading the bar code or the two-dimensional code, and transmitting the information written in the bar code or the two-dimensional code to the radiological imaging apparatus.

9. A radiological imaging system comprising:
the radiological imaging apparatus according to claim 1; and
a console configured to generate a radiological image in accordance with the image data transmitted from the radiological imaging apparatus,
wherein the radiological imaging apparatus attaches the information to the image data, and transmits the image data to the console, and
the console associates the generated radiological image with imaging order information corresponding to the information attached to the image data on which the radiological image is based.
